(19) United States
(12) Patent Application Publication
(10) Pub. No.: US 2024/0124656 A1
(43) Pub. Date: Apr. 18, 2024

(54) CONDUCTIVE HYDROGEL COMPRISING MUSSEL ADHESIVE PROTEIN AND PREPARATION METHOD THEREOF

(71) Applicant: POSTECH RESEARCH AND BUSINESS DEVELOPMENT FOUNDATION, Pohang-si (KR)

(72) Inventors: Hyung Joon Cha, Pohang-si (KR); Jae Yun Lee, Pohang-si (KR); Jin Young Yun, Pohang-si (KR); Hyun Tack Woo, Pohang-si (KR)

(21) Appl. No.: 18/474,363

(22) Filed: Sep. 26, 2023

(30) Foreign Application Priority Data
Oct. 4, 2022 (KR) 10-2022-0126341

(51) Int. Cl. C08H 1/00; C07K 14/435; C08B 37/08

(52) U.S. Cl. CPC C08H 1/00; C07K 14/43504; C08B 37/0072

(57) ABSTRACT

The present disclosure relates to a conductive hydrogel including a mussel adhesive protein, a liquid metal, and hyaluronic acid and a preparation method thereof.

### CROSS-REFERENCE TO RELATED APPLICATIONS

This application is based on and claims priority from Korean Patent Application No. 10-2022-0126341, filed on Oct. 4, 2022, with the Korean Intellectual Property Office, the disclosure of which is incorporated herein in its entirety by reference.

### TECHNICAL FIELD

The present disclosure relates to a conductive hydrogel including a mussel adhesive protein and a preparation method thereof.

### BACKGROUND

A mussel adhesive protein is a protein rich in 3,4-dihydroxyphenylalanine (DOPA) residues, which form hydrogen bonds or covalent bonds with nucleophiles such as amine groups, thiol groups, and hydroxyl groups on the surface of tissues to enable surface adhesion. These DOPA residues form metal-catechol complexes with metal elements to show excellent adhesive strength even on metal surfaces as well as on tissue surfaces, and also impart excellent mechanical properties to materials made of protein, such as mussel byssus. The mussel adhesive protein has not only excellent adhesive strength in water through the DOPA residues, but also excellent biocompatibility and biodegradability as a bio-derived polymer, and thus has been extensively studied as a biomaterial for medical use.

Specifically, a conventional mussel adhesive protein-based hydrogel is formed through crosslinking of 3,4-dihydroxyphenylalanine (DOPA), which is an amino acid present in the mussel adhesive protein, and there is a method of complete oxidation crosslinking of DOPA and a method of crosslinking through iron ions.

However, in the case of complete oxidative crosslinking, DOPA, a key amino acid that enables the mussel adhesive protein to exhibit adhesive strength, is oxidized to completely lose adhesion, and even in a crosslinking method using iron ions, DOPA to contribute to adhesion contributes to crosslinking, and thus the adhesive strength is remarkably reduced.

Therefore, a conventional mussel adhesive protein-based hydrogel has limitations in applications because the mussel adhesive protein may not form an interface with adhesive strength on a desired surface.

### SUMMARY

The present inventors developed a method for forming a hydrogel with excellent adhesive strength and conductivity by hydrogeling a composition for preparing a hydrogel containing a conductive substance using electrical oxidation in an environment required for adhesive strength, to prepare a hydrogel imparted with conductivity while overcoming the limitations of the related art as described above.

The present disclosure has been made in an effort to provide a conductive hydrogel including a mussel adhesive protein having adhesive strength and conductivity.

In addition, the present disclosure has also been made in an effort to provide a preparation method of the hydrogel.

An exemplary embodiment of the present disclosure provides a conductive hydrogel including a mussel adhesive protein, a liquid metal and hyaluronic acid.

The liquid metal may be a liquid metal consisting of gallium-indium nanoparticles.

The liquid metal may include 65 to 80 wt % of gallium and 20 to 35 wt % of indium.

The hydrogel may be a coacervate-based hydrogel formed by crosslinking nanoparticles coated with hyaluronic acid on the outside of the liquid metal; and a mussel adhesive protein.

The nanoparticles may be 20 to 3000 nm.

The mussel adhesive protein may consist of at least one amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15.

The mussel adhesive protein may be a mussel adhesive protein in which tyrosine residues are converted into a catechol compound; the catechol compound is introduced to the surface of the mussel adhesive protein; or both thereof.

The catechol compound may be at least one selected from the group consisting of 3,4-dihydroxyphenylalanine (DOPA), Dopa o-quinone, 2,4,5-trihydroxyphenylalanine (TOPA), Topaquinone, and derivatives thereof.

Another exemplary embodiment of the present disclosure provides a preparation method of a conductive hydrogel including (a) preparing a first mixed solution by mixing a solution containing hyaluronic acid (HA) and a liquid metal; (b) preparing a second mixed solution by mixing the first mixed solution and a solution containing a mussel adhesive protein; and (c) preparing a hydrogel by applying electrical stimulation to the second mixed solution.

In step (a), the solution containing hyaluronic acid may contain 0.1 to 2 wt % of hyaluronic acid based on the total weight of the mixed solution.

In step (a), the liquid metal may contain 1 to 5 wt % based on the total weight of the mixed solution.

In step (b), the solution containing the mussel adhesive protein may contain 0.3 to 2 wt % of the mussel adhesive protein based on the total weight of the solution.

In step (b), the second mixed solution may be prepared by mixing the first mixed solution and the solution containing the mussel adhesive protein in a concentration ratio of 1:1 to 1:3.

In step (c), catechols may be crosslinked by electrical stimulation to form a hydrogel.

According to the exemplary embodiments of the present disclosure, since the hydrogel includes the mussel adhesive protein, the liquid metal, and the hyaluronic acid to not only have excellent adhesive strength and mechanical strength, but also have conductivity, it is possible to apply conductivity to the surface without using additional adhesives.

Therefore, according to the exemplary embodiments of the present disclosure, the hydrogel may be used for wearable electronic products, artificial skin, supercapacitor robotics, energy storage materials, bioelectrodes, implantable current measurement biosensors, electrical stimulation drug release devices, and neural prostheses.

It should be understood that the effects of the present disclosure are not limited to the effects, but include all effects that may be deduced from the detailed description of the present disclosure or configurations of the present disclosure described in appended claims.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1A to 1D is an image of confirming a process of producing a hydrogel prepared according to Example 1 of the present disclosure, in which FIG. 1A is an image of confirming a first mixed solution prepared by mixing a solution containing hyaluronic acid (HA) and a liquid metal, FIG. 1B is an image of a solution containing a mussel adhesive protein, FIG. 1C is an image of confirming a coacervate formed by crosslinking a nanoparticle coated with hyaluronic acid on the outside of the liquid metal with the mussel adhesive protein, and FIG. 1D is an image of confirming that a hydrogel is formed after applying electrical stimulation to the coacervate.

FIG. 2A to 2D is a transmission electron microscopy (TEM) image of a nanoparticle coated with hyaluronic acid on the outside of the liquid metal, in which FIG. 2A is a TEM image of liquid metal-nanoparticles when has used distilled water instead of a hyaluronic acid solution, FIG. 2B is a TEM image of liquid metal nanoparticles coated with a 0.1 wt % hyaluronic acid solution, FIG. 2C is a TEM image of liquid metal nanoparticles coated with a 0.3 wt % hyaluronic acid solution, and FIG. 2D is a TEM image of liquid metal nanoparticles coated with a 0.5% hyaluronic acid solution.

FIG. 3 illustrates a result of confirming the sizes of the nanoparticles confirmed in FIG. 2A to 2D.

FIG. 4A to 4C is a graph showing dynamic moduli of a coacervate and a hydrogel containing liquid metal nanoparticles coated with 0.1 wt %, 0.3 wt % and 0.5 wt % of hyaluronic acid solutions and a mussel adhesive protein, in which FIG. 4A shows a graph of a 0.1 wt % hyaluronic acid solution, FIG. 4B shows a graph of a 0.3 wt % hyaluronic acid solution, and FIG. 4C shows a graph of a 0.5 wt % hyaluronic acid solution.

FIG. 5 is a result of confirming the adhesive strengths of a coacervate prepared according to Example 3 of the present disclosure, a hydrogel, and after immersion in PBS for 2 hours after forming the hydrogel.

FIG. 6 is a schematic diagram and a result of confirming the impedance of the hydrogel prepared according to Example 3 of the present disclosure.

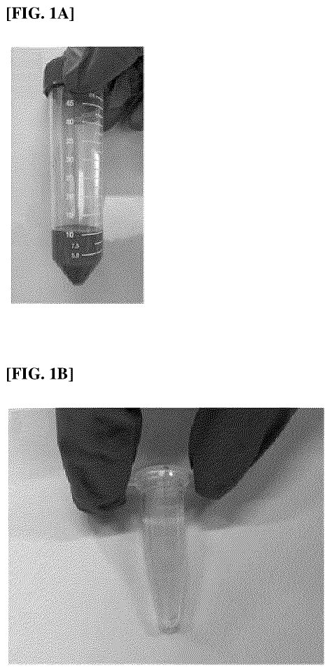

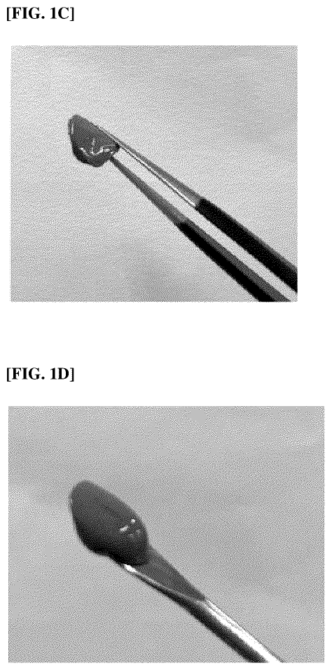

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

In the following description, only parts required to understand exemplary examples of the present disclosure will be described, and it should be noted that the description of other parts will be omitted within a range without departing from the gist of the present disclosure.

Terms and words used in the present specification and claims should not be interpreted as being limited to typical or dictionary meanings, but should be interpreted as meanings and concepts which comply with the technical spirit of the present disclosure, based on the principle that the present inventor may appropriately define the concepts of the terms to describe his/her own invention in the best manner. Therefore, the exemplary examples described in the present specification and the configurations illustrated in the drawings are merely the most preferred exemplary example of the present disclosure and are not intended to represent all of the technical ideas of the present disclosure, and thus, it should be understood that various equivalents and modifications capable of replacing the exemplary examples at the time of this application.

Hereinafter, the present disclosure will be described in detail.

Conductive Hydrogel

The present disclosure provides a conductive hydrogel including a mussel adhesive protein, a liquid metal and hyaluronic acid.

The hydrogel according to the present disclosure is characterized by producing a hydrogel containing a mussel adhesive protein using a known DOPA-iron ion bond and simultaneously providing a hydrogel with conductivity by including a liquid metal.

In the present disclosure, the “liquid metal” refers to a metallic material that maintains a liquid phase even at room temperature due to its low melting point, and may be specifically any one selected from the group consisting of mercury, cesium, radium, francium, rubidium, and eutectic Ga—In alloy (EGaIn), more specifically an eutectic Ga—In alloy with relatively low toxicity when used in the body, and the eutectic Ga—In alloy may refer to a Ga—In nanoparticle.

The Ga—In liquid metal includes 65 to 80 wt % of gallium and 20 to 35 wt % of indium, and when including gallium and indium within the range, the Ga—In liquid metal may exhibit high electrical and thermal conductivity while maintaining mechanical strength at a level usable in the body.

The hydrogel may be a coacervate-based hydrogel formed by crosslinking nanoparticles coated with hyaluronic acid on the outside of the liquid metal; and a mussel adhesive protein.

In the present disclosure, the “coacervate” refers to a type of colloid formed by linking a cationic protein and an anionic polymer, and may be formed by mixing the cationic protein and the anionic polymer.

In the present disclosure, the cationic protein may be a mussel adhesive protein or a mutant thereof, and the anionic polymer may be hyaluronic acid.

The average molecular weight of hyaluronic acid used as the ionic polymer is not limited thereto, but may be 1 kDa to 8000 kDa, specifically 300 kDa to 1000 kD, more specifically 500 kDa to 800 kDa, and the coacervates may be easily formed within the molecular weight range.

When hyaluronic acid is coated on the outside of the liquid metal, oxidation and re-aggregation of the liquid metal (e.g., gallium-indium nanoparticles) may be prevented.

The nanoparticle consisting of hyaluronic acid coated on the outside of the liquid metal may have a size of 20 to 3000 nm, specifically 30 to 1500 nm, and more specifically 40 to 500 nm.

The mussel adhesive protein is a protein derived from the mussel byssus, and preferably a mussel adhesive protein derived from Mytilus edulis, Mytilus galloprovincialis or Mytilus coruscus or its mutant, but is not limited thereto.

The mussel adhesive protein of the present disclosure may include Mytilus edulis foot protein (Mefp)-1, Mytilus galloprovincialis foot protein (Mgfp)-1, Mytilus coruscus foot protein (Mcfp)-1, Mefp-2, Mefp-3, Mgfp-3, and Mgfp-5 derived from each of the mussel species or mutants thereof, preferably a protein selected from the group consisting of foot protein (fp)-1 (SEQ ID NO: 1), fp-2 (SEQ ID NO: 4), fp-3 (SEQ ID NO: 5), fp-4 (SEQ ID NO: 6), fp-5 (SEQ ID NO: 7), and fp-6 (SEQ ID NO: 8), a fusion protein in which two or more proteins are linked, or a mutant of the protein, but is not limited thereto.

In addition, the mussel adhesive protein of the present disclosure includes all mussel adhesive proteins described in International Publication No. WO2006/107183 or WO2005/092920. Preferably, the mussel adhesive protein may include a fusion protein selected from the group consisting of fp-151 (SEQ ID NO: 9), fp-131 (SEQ ID NO: 10), fp-353 (SEQ ID NO: 11), fp-153 (SEQ ID NO: 12) and fp-351 (SEQ ID NO: 13), but is not limited thereto.

In addition, the mussel adhesive protein of the present disclosure may include a polypeptide in which decapeptides (SEQ ID NO: 2) repeated about 80 times in fp-1 are continuously linked to each other 1 to 12 times or more.

In addition, the mussel adhesive protein of the present disclosure may include a polypeptide in which decapeptides (SEQ ID NO: 2) repeated about 80 times in fp-1 are continuously linked to each other 1 to 12 times or more. Preferably, the mussel adhesive protein may be an fp-1 variant polypeptide (SEQ ID NO: 3) in which decapeptides of SEQ ID NO: 2 are continuously linked to each other 12 times, but is not limited thereto.

In addition, the mussel adhesive protein of the present disclosure may be an fp-151 variant mutant (SEQ ID NO: 15), but is not limited thereto. Compared to SEQ ID NO: 9, the protein sequence represented by SEQ ID NO: 15 is a sequence excluding a linker sequence and the like. Specifically, the protein sequence represented by SEQ ID NO: 15 is a fusion protein sequence in which the sequence of Mgfp-5 represented by SEQ ID NO: 16 is fused between the fp-1 variant sequences represented by SEQ ID NO: 14. More specifically, the mussel adhesive protein may consist of at least one amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and SEQ ID NO: 16.

In the present disclosure, the mussel adhesive protein may also be modified within the range including a conserved amino acid sequence capable of maintaining the characteristics of the above-mentioned mussel adhesive proteins. That is, the scope of the present disclosure may include amino acid sequences having sequence identify of 70% or more, preferably 80% or more, and much more preferably 90% or more, that is, 95%, 96%, 97%, 98%, and 99% or more with the amino acid sequences represented by SEQ ID NOs that exhibit substantially equivalent effects.

The mussel adhesive protein may be a mussel adhesive protein in which tyrosine residues are converted into a catechol compound; the catechol compound is introduced to the surface of the mussel adhesive protein; or both thereof.

In the mussel adhesive protein of the present disclosure, tyrosine residues are preferably converted into catechol compounds, and 10 to 100% of the total tyrosine residues are preferably converted into catechol compounds. The proportion of tyrosine in the entire amino acid sequence of most mussel adhesive proteins may be about 1 to 50%. The tyrosine in the mussel adhesive protein may be converted to DOPA, a catechol compound added with an OH group through a hydration process.

However, in mussel adhesive proteins produced in E. coli, since tyrosine residues are not converted, it is preferred to perform a modification reaction of converting tyrosine into DOPA by a separate enzymatic and chemical treatment method. A method known in the art may be used to modify the tyrosine residues included in the mussel adhesive protein with DOPA, and is not particularly limited.

The catechol compound is a compound containing a dihydroxy group, and refers to a compound that imparts adhesive strength to the mussel adhesive protein through a crosslinking action. Specifically, the catechol compound may be at least one selected from the group consisting of 3,4-dihydroxyphenylalanine (DOPA), Dopa o-quinone, 2,4,5-trihydroxyphenylalanine (TOPA), Topaquinone, and derivatives thereof.

In the present disclosure, mutants of the mussel adhesive protein may be preferably produced by including additional sequences in a carboxyl terminal or amino terminal of the mussel adhesive protein or converting some amino acids into other amino acids under the premise of maintaining the adhesive strength of the mussel adhesive protein. More preferably, a polypeptide consisting of 3 to 25 amino acids including RGD is linked to the carboxyl terminal or amino terminal of the mussel adhesive protein, or 1% to 100%, preferably 5% to 100% of the total number of tyrosine residues constituting the mussel adhesive protein may be converted into 3,4-dihydroxyphenyl-L-alanine (DOPA).

The mussel adhesive protein is not limited thereto, but preferably may be mass-produced by genetic engineering by inserting a gene into a conventional vector produced to be able to express an external gene. The vector may be appropriately selected or newly produced according to the type and characteristics of a host cell for producing the protein. A method of transforming the vector into a host cell and a method of producing a recombinant protein from a transformant may be easily performed by conventional methods. Methods such as selection, construction, and transformation of the vector, and expression of recombinant proteins may be easily performed by those skilled in the art, and some modifications of conventional methods are also included in the present disclosure.

The conductive hydrogel according to the present disclosure may be usefully applied to biomedical applications requiring bioadhesion. For example, the conductive hydrogel may be used as a biomaterial-based tissue adhesive and may be applied as an adhesive for fixing a device mounted in a living body for in vivo signal transmission and drug administration.

Preparation Method of Conductive Hydrogel

Further, the present disclosure provides a preparation method of a conductive hydrogel including (a) preparing a first mixed solution by mixing a solution containing hyaluronic acid (HA) and a liquid metal; (b) preparing a second mixed solution by mixing the first mixed solution and a solution containing a mussel adhesive protein; and (c) preparing a hydrogel by applying electrical stimulation to the second mixed solution.

Step (a) is a step of mixing a solution containing hyaluronic acid (HA) and a liquid metal to prepare a first mixed solution containing nanoparticles coated with hyaluronic acid on the outside of the liquid metal.

In step (a), the solution containing hyaluronic acid may contain 0.1 to 2 wt %, specifically 0.1 to 1 wt % of hyaluronic acid based on the total weight of the first mixed solution, and when the hyaluronic acid is included within the range, the hyaluronic acid is coated on the outside of the liquid metal at an appropriate thickness to form a coacervate with the mussel adhesive protein. On the other hand, at a concentration of less than 0.1 wt %, the formation of a hyaluronic acid coating on the outer surface of the liquid metal is not conspicuous, and in more than 2 wt %, due to hyaluronic acid not coated on the outer surface of the liquid metal, the conductivity of the mixed solution may be hindered.

In step (a), the liquid metal may be included in 1 to 5 wt %, specifically 1 to 3 wt % based on the total weight of the first mixed solution, and when the liquid metal is included within the range, nanoparticles having an appropriate size may be formed. Conductivity may not be exhibited at a concentration of less than 1 wt %, and formation of nanoparticles due to ultrasound may be inhibited at a concentration of more than 5 wt %.

Step (b) is a step of mixing the first mixed solution containing nanoparticles coated with hyaluronic acid on the outside of liquid metal and a solution containing a mussel adhesive protein to prepare a second mixed solution.

Specifically, step (b) may be a step of crosslinking the nanoparticles coated with hyaluronic acid on the outside of the liquid metal with the mussel adhesive protein through electrostatic attraction to form a coacervate.

The mussel adhesive protein may be a mussel adhesive protein in which tyrosine residues are converted into a catechol compound; the catechol compound is introduced to the surface of the mussel adhesive protein; or both thereof. The mussel adhesive protein of the present disclosure is preferably a mussel adhesive protein in which tyrosine residues are converted into a catechol compound.

That is, step (b) may be a step of crosslinking the nanoparticles coated with hyaluronic acid on the outside of the liquid metal with the mussel adhesive protein in which the tyrosine residues are converted into the catechol compound to form a coacervate.

In step (b), the solution containing the mussel adhesive protein may be included in 0.1 to 5 wt %, specifically 0.3 to 1 wt % of the mussel adhesive protein based on the total weight of the solution, and when the mussel adhesive protein is included within the range, a hydrogel having usable adhesive strength may be formed. When the concentration of less than 0.1 wt % is used, the usable adhesive force is not formed, and when the concentration of more than 5 wt % is used, conductivity may be impaired.

In step (b), the second mixed solution may be prepared by mixing the first mixed solution and the solution containing the mussel adhesive protein in a concentration ratio of 1:1 to 1:3, specifically 1:2. At this time, when the solution containing the mussel adhesive protein is mixed in the ratio range based on the first mixed solution, the coacervate may be easily formed.

Step (c) is a step of preparing a hydrogel by applying electrical stimulation to the coacervate formed in step (b).

Specifically, in step (c), the catechols of the mussel adhesive protein in which the tyrosine residues in the coacervate formed in step (b) are converted into the catechol compound are crosslinked by electrical stimulation to form a hydrogel.

The electrical stimulation is performed by applying a voltage of 0.1 V to 30 V, specifically 0.5 V to 10 V, more specifically 1 V to 8 V to the second mixed solution for 10 seconds to 10 minutes using a DC power supply device in the air or in the body environment.

The description of the mussel adhesive protein, the liquid metal, the coacervate, and the hydrogel mentioned in the preparation method of the conductive hydrogel of the present disclosure is the same as described in the conductive hydrogel.

Therefore, the conductive hydrogel according to the present disclosure may be usefully applied as adhesives of wearable electronic products, artificial skin, supercapacitor robotics, energy storage materials, bioelectrodes, implantable current measurement biosensors, electrical stimulation drug release devices, and neural prostheses.

The above description just illustrates the technical spirit of the present disclosure and various changes and modifications may be made by those skilled in the art to which the present disclosure pertains without departing from an essential characteristic of the present disclosure. Accordingly, the various exemplary embodiments disclosed in the present disclosure are not intended to limit the technical spirit but describe the present disclosure and the technical spirit of the present disclosure is not limited by the following exemplary embodiments. The protective scope of the present disclosure should be construed based on the following claims, and all the techniques in the equivalent scope thereof should be construed as falling within the scope of the present disclosure.

### Preparation Example

### Preparation Example 1. Preparation of Solution Containing Hyaluronic Acid

Hyaluronic acid (Mw: 732 kDa, SK Bioland Co., Ltd.) was dissolved in distilled water at a temperature of 25° C. to prepare three solutions with different concentrations (0.1 wt %, 0.3 wt % and 0.5 wt %).

### Preparation Example 2: Preparation of Solution Containing Mussel Adhesive Protein

(1) Preparation of Mussel Adhesive Protein

A mussel adhesive protein fp-151 of SEQ ID NO: 9 was produced in E. coli by synthesizing a fp-1 variant consisting of 6 decapeptides so that a decapeptide consisting of 10 amino acids that were repeated about 80 times in a mussel adhesive protein fp-1 naturally existing may be expressed in E. coli and inserting a Mgfp-5 gene (Genbank No. AAS00463 or AY521220) between the two fp-1 variants (D. S. Hwang et. al., Biomaterials 28, 3560-3568, 2007). Next, a mussel adhesive protein was prepared in which a catechol DOPA residue was introduced into a mussel adhesive protein fp-151 of SEQ ID NO: 9 by converting tyrosine of the mussel adhesive protein into DOPA through an in vitro enzymatic reaction using mushroom tyrosinase.

(2) Preparation of Solution Containing Mussel Adhesive Protein

A solution containing 0.8 wt % of a mussel adhesive protein was prepared by mixing 80 mg of the prepared mussel adhesive protein in 10 mL of distilled water.

### EXAMPLE

### Example 1: In the Case of 0.1 wt % of Hyaluronic Acid

150 mg of the gallium-indium liquid metal (Ga: 75.5 wt %, In: 24.5 wt %, Alfa Aesar) prepared in Preparation Example 3 was mixed with 10 mL of the hyaluronic acid solution (0.1 wt %) prepared in Preparation Example 1, sonicated for 20 minutes, and then centrifuged at a rate of 1000 rpm to take a supernatant (first mixed solution). The supernatant was mixed with 10 mL of the solution containing the mussel adhesive protein prepared in Preparation Example 2 to prepare a second mixed solution, and the second mixed solution was centrifuged at a rate of 2000 rpm to obtain a coacervate from a precipitate. The coacervate was applied with a voltage of 1 V for 10 seconds using a DC power supply device in an atmospheric environment, and a hydrogel was prepared through a reaction time of about 10 minutes.

### Example 2. In the Case of 0.3 wt % of Hyaluronic Acid

A hydrogel was prepared in the same manner and conditions as in Example 1, except for using 0.3 wt % of a hyaluronic acid solution instead of 0.1 wt % of the hyaluronic acid solution in Example 1.

### Example 3. In the Case of 0.5 wt % of Hyaluronic Acid

A hydrogel was prepared in the same manner and conditions as in Example 1, except for using 0.5 wt % of a hyaluronic acid solution instead of 0.1 wt % of the hyaluronic acid solution in Example 1.

### Comparative Example

### Comparative Example 1

150 mg of solid silver nano-powder (Silver, Sigma Aldrich, 576832) was mixed with 10 mL of the hyaluronic acid solution (0.5 wt %) prepared in Preparation Example 1, sonicated for 20 minutes, and then centrifuged at a rate of 1000 rpm to take a supernatant. The supernatant was mixed with 10 mL of the solution containing the mussel adhesive protein prepared in Preparation Example 2 to prepare a mixed solution, and the mixed solution was centrifuged at a rate of 2000 rpm to obtain a coacervate from a precipitate. The coacervate was applied with a voltage of 1 V for 10 seconds using a DC power supply device and reacted for 10 minutes, but a hydrogel was not formed.

### Comparative Example 2

150 mg of graphene oxide (Graphene, Sigma Aldrich, 900561) as a conductive material was mixed with 10 mL of the hyaluronic acid solution (0.5 wt %) prepared in Preparation Example 1, sonicated for 20 minutes, and then centrifuged at a rate of 1000 rpm to take a supernatant. The supernatant was mixed with 10 mL of the solution containing the mussel adhesive protein prepared in Preparation Example 2 to prepare a mixed solution, and the mixed solution was centrifuged at a rate of 2000 rpm to obtain a coacervate from a precipitate. The coacervate was applied with a voltage of 1 V for 10 seconds using a DC power supply device and reacted for 10 minutes, and then a hydrogel was prepared. However, the graphene oxide had very severe cytotoxicity, so that there was a problem that the graphene oxide could not be used in the body.

### Test Example

### Test Example 1. Confirmation of Nanoparticles Coated with Hyaluronic Acid on Outside of Liquid Metal

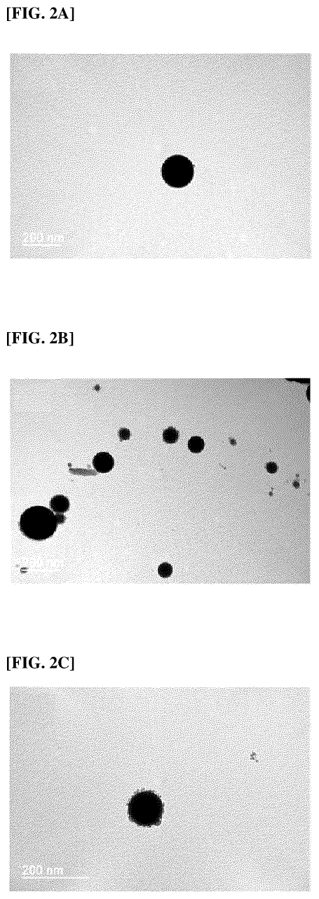

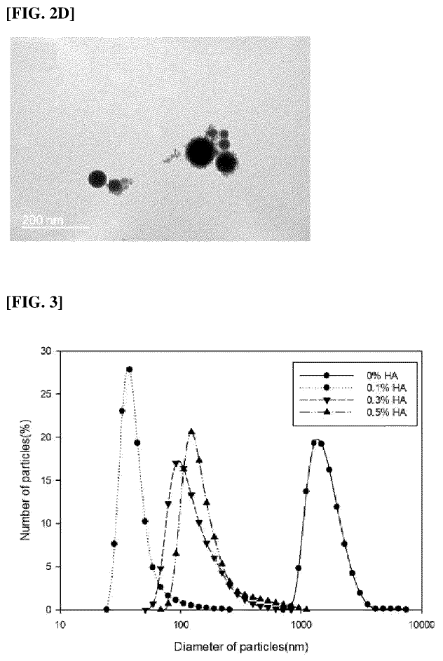

FIG. 2A to 2D is a transmission electron microscopy (TEM) image of a nanoparticle coated with hyaluronic acid on the outside of the liquid metal, in which FIG. 2A is a TEM image of liquid metal-nanoparticles when has used distilled water instead of a hyaluronic acid solution, FIG. 2B is a TEM image of liquid metal nanoparticles coated with a 0.1 wt % hyaluronic acid solution, FIG. 2C is a TEM image of liquid metal nanoparticles coated with a 0.3 wt % hyaluronic acid solution, and FIG. 2D is a TEM image of liquid metal nanoparticles coated with a 0.5% hyaluronic acid solution.

Referring to FIGS. 2A to 2D, it was confirmed that the coating thickness coated on the liquid metal nanoparticle increased as the concentration of hyaluronic acid increased.

### Test Example 2. Particle Size Analysis of Nanoparticles Coated with Hyaluronic Acid on Outside of Liquid Metal

For the nanoparticles identified in FIG. 2A to 2D, particle size analysis was confirmed by Dynamic Light Scattering by Malvern Corporation, and the results were shown in FIG. 3.

Referring to FIG. 3, it may be seen that the size of the liquid metal nanoparticles increased as the concentration of hyaluronic acid increased.

In addition, when hyaluronic acid was not coated on the outside of the liquid metal nanoparticles (FIG. 2A), it was confirmed that oxidation and re-aggregation of the gallium-indium nanoparticles occurred, so that the particle size was larger than that of the liquid metal nanoparticles coated with hyaluronic acid (FIGS. 2B to 2D).

### Test Example 3. Confirmation of Mechanical Properties

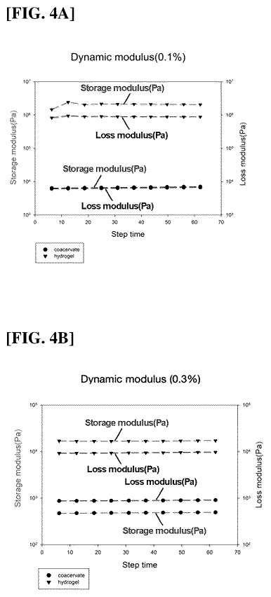

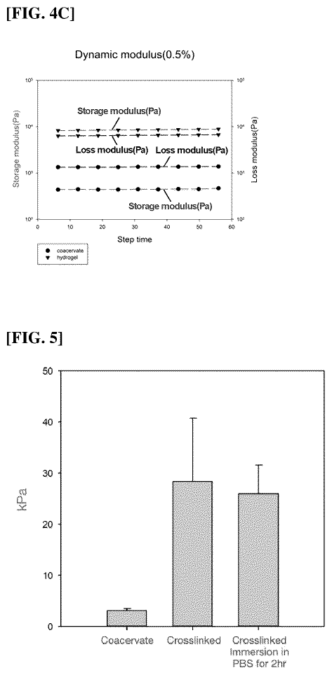

Mechanical properties were measured using a Rheometer system (DHR-2, TA instruments) at a frequency of 10.0 rad/s with a force of 1% for 60 seconds at 25° C., and the results were illustrated in FIG. 4A to 4C.

Specifically, FIG. 4A to 4C is a graph showing dynamic moduli of a coacervate and a hydrogel containing liquid metal nanoparticles coated with 0.1 wt %, 0.3 wt % and 0.5 wt % of hyaluronic acid solutions and a mussel adhesive protein, in which FIG. 4A shows a graph of a 0.1 wt % hyaluronic acid solution, FIG. 4B shows a graph of a 0.3 wt % hyaluronic acid solution, and FIG. 4C shows a graph of a 0.5 wt % hyaluronic acid solution.

In FIGS. 4A to 4C, a circle mark indicates a coacervate, an inverted triangle mark indicates a physical property of a hydrogel, and a red dotted line indicates a storage modulus and a blue dotted line indicates a loss modulus. After the formation of the hydrogel by electrical stimulation, it was confirmed that a phase change from a liquid phase to a solid phase and an increase in strength occurred through crosslinking through a change in elastic modulus tendency and a change in mechanical strength.

### Test Example 4. Confirmation of Adhesive Strength

Each sample was prepared by attaching 1 cm 2 of pig skin among biomaterials to an FTO glass substrate with the coacervate prepared in Example 3, the hydrogel, and the hydrogel after immersion in PBS for 2 hours after forming the hydrogel. The adhesive strength of the sample was confirmed using Instron (Instron model 3344, Universa) by pulling the FTO glass substrate and the pig skin attached to the FTO glass substrate with a force of 1 N/s, and the results were illustrated in FIG. 5.

Referring to FIG. 5, it was confirmed that there was a statistically significant difference in adhesive strength between a coacervate and a hydrogel (“Crosslinked” and “Crosslinked Immersion in PBS for 2 hr”).

On the other hand, since it may be confirmed that the adhesive strength of the hydrogel is maintained even after being exposed to an underwater environment, it may be confirmed that the underwater adhesive strength to an interface is formed through crosslinking by electrical stimulation.

### Test Example 5. Confirmation of Conductivity

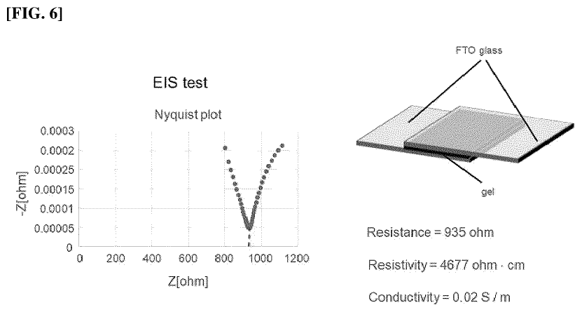

The impedance of a substrate on which the hydrogel was formed at a thickness of 0.5 cm² between two FTO glass substrates was measured using a Potentiostat (VSP-300, BioLogic), and the results were illustrated in FIG. 6.

Specifically, FIG. 6 is a result of confirming the impedance of the hydrogel prepared according to Example 3 of the present disclosure and the specific resistance and conductivity in the DC power supply accordingly.

Referring to FIG. 6, it may be seen that the conductivity of the hydrogel is 0.02 S/m, which shows excellent conductivity among hydrogels using biocompatible materials.

The present disclosure has been described with reference to the preferred exemplary examples of the present disclosure, but those skilled in the art will understand that the present disclosure may be variously modified and changed without departing from the spirit and the scope of the present disclosure which are defined in the appended claims.

Accordingly, the technical scope of the present disclosure should not be limited to the contents disclosed in the detailed description of the specification but should be defined only by the claims.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A conductive hydrogel comprising:
a mussel adhesive protein;
a liquid metal; and
hyaluronic acid.

2. The conductive hydrogel of claim 1, wherein the liquid metal is a liquid metal consisting of gallium-indium nanoparticles.

3. The conductive hydrogel of claim 1, wherein the liquid metal includes 65 to 80 wt % of gallium and 20 to 35 wt % of indium.

4. The conductive hydrogel of claim 1, wherein the hydrogel is a coacervate-based hydrogel formed by crosslinking nanoparticles coated with hyaluronic acid on the outside of the liquid metal; and a mussel adhesive protein.

5. The conductive hydrogel of claim 4, wherein the nanoparticles are 20 to 3000 nm.

6. The conductive hydrogel of claim 1, wherein the mussel adhesive protein consists of at least one amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and SEQ ID NO: 15.

7. The conductive hydrogel of claim 1, wherein the mussel adhesive protein is a mussel adhesive protein in which tyrosine residues are converted into a catechol compound; the catechol compound is introduced to the surface of the mussel adhesive protein; or both thereof.

8. The conductive hydrogel of claim 7, wherein the catechol compound is at least one selected from the group consisting of 3,4-dihydroxyphenylalanine (DOPA), Dopa o-quinone, 2,4,5-trihydroxyphenylalanine (TOPA), Topaquinone, and derivatives thereof.

9. A preparation method of a conductive hydrogel comprising:
(a) preparing a first mixed solution by mixing a solution containing hyaluronic acid (HA) and a liquid metal;
(b) preparing a second mixed solution by mixing the first mixed solution and a solution containing a mussel adhesive protein; and
(c) preparing a hydrogel by applying electrical stimulation to the second mixed solution.

10. The preparation method of the conductive hydrogel of claim 9, wherein in step (a), the solution containing hyaluronic acid contains 0.1 to 2 wt % of hyaluronic acid based on the total weight of the mixed solution.

11. The preparation method of the conductive hydrogel of claim 9, wherein in step (a), the liquid metal is included in 1 to 5 wt % based on the total weight of the mixed solution.

12. The preparation method of the conductive hydrogel of claim 9, wherein in step (b), the solution containing the mussel adhesive protein contains 0.3 to 2 wt % of the mussel adhesive protein based on the total weight of the solution.

13. The preparation method of the conductive hydrogel of claim 9, wherein in step (b), the second mixed solution is prepared by mixing the first mixed solution and the solution containing the mussel adhesive protein in a concentration ratio of 1:1 to 1:3.

14. The preparation method of the conductive hydrogel of claim 9, wherein the mussel adhesive protein is a mussel adhesive protein in which tyrosine residues are converted into a catechol compound; the catechol compound is introduced to the surface of the mussel adhesive protein; or both thereof.

15. The preparation method of the conductive hydrogel of claim 14, wherein in step (c), catechols are crosslinked by electrical stimulation to form a hydrogel.


SEQUENCE LISTING (16 sequences)

SEQ ID NO: 1 - protein, 800 aa, synthetic construct
  REGION 1..800: note = fp-1
AKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYK

SEQ ID NO: 2 - protein, 10 aa, synthetic construct
  REGION 1..10: note = fp-1 variant
AKPSYPPTYK

SEQ ID NO: 3 - protein, 120 aa, synthetic construct
  REGION 1..120: note = fp-1 variant
AKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYK

SEQ ID NO: 4 - protein, 472 aa, synthetic construct
  REGION 1..472: note = fp-2
LFSFFLLLTCTQLCLGTNRPDYNDDEEDDYKPPVYKPSPSKYRPVNPCLKKPCKYNGVCKPRGGSYKCFCKGGYYGYNCNLKNACKPNQCKNKSRCVPVGKTFKCVCRNGNFGRLCEKNVCSPNPCKNNGKCSPLGKTGYKCTCSGGYTGPRCEVHACKPNPCKNKGRCFPDGKTGYKCRCVDGYSGPTCQENACKPNPCSNGGTCSADKFGDYSCECRPGYFGPECERYVCAPNPCKNGGICSSDGSGGYRCRCKGGYSGPTCKVNVCKPTPCKNSGRCVNKGSSYNCICKGGYSGPTCGENVCKPNPCQNRGRCYPDNSDDGFKCRCVGGYKGPTCEDKPNPCNTKPCKNGGKCNYNGKIYTCKCAYGWRGRHCTDKAYKPNPCVVSKPCKNRGKCIWNGKAYRCKCAYGYGGRHCTKKSYKKNPCASRPCKNRGKCTDKGNGYVCKCARGYSGRYCSLKSPPSYDDDEY

SEQ ID NO: 5 - protein, 50 aa, synthetic construct
  REGION 1..50: note = fp-3
PWADYYGPKYGPPRRYGGGNYNRYGRRYGGYKGWNNGWKRGRWGRKYYGS

SEQ ID NO: 6 - protein, 750 aa, synthetic construct
  REGION 1..750: note = fp-4
YGRRYGEPSGYANIGHRRYYERAISFHRHSHVHGHHLLHRHVHRHSVLHGHVHMHRVSHRIMHRHRVLHGHVHRHRVLHNHVHRHSVLHGHVHRHRVLHRHVHRHNVLHGHVHRHRVLHKHVHNHRVLHKHLHKHQVLHGHVHRHQVLHKHVHNHRVLHKHLHKHQVLHGHVHTHRVLHKHVHKHRVLHKHLHKHQVLHGHIHTHRVLHKHLHKHQVLHGHVHTHRVLHKHVHKHRVLHKHLHKHQVLHGHVHMHRVLHKHVHKHRVLHKHVHKHHVVHKHVHSHRVLHKHVHKHRVEHQHVHKHHVLHRHVHSHHVVHSHVHKHRVVHSHVHKHNVVHSHVHRHQILHRHVHRHQVVHRHVHRHLIAHRHIHSHQAAVHRHVHTHFEGNFNDDGTDVNLRIRHGIIYFGGNTYRLSGGRRRFMTLWQECLESYGDSDECFVQLLEGNQHLFTVVQGHHSTSFRSDLSNDLHPDNNIEQIANDHVNDIAQSTDGDINDFADTHYNDVAPIADVHVDNIAQTADNHVKNIAQTAHHHVNDVAQIADDHVNDIGQTAYDHVNNIGQTADDHVNDIAQTADDHVNAIAQTADDHVNAIAQTADDHVNDIGDTANSHIVRVQGVAKNHLYGINKAIGKHIQHLKDVSNRHIEKLNNHATKNLLQSALQHKQQTIEREIQHKRHLSEKEDINLQHENAMKSKVSYDGPVFNEKVSVVSNQGSYNEKVPVLSNGGGYNGKVSALSDQGSYNEGYAY

SEQ ID NO: 7 - protein, 82 aa, synthetic construct
  REGION 1..82: note = fp-5
KHHHHHHSSEEYKGGYYPGNTYHYHSGGSYHGSGYHGGYKGKYYGKAKKYYYKYKNSGKYKYLKKARKYHRKGYKKYYGGSS

SEQ ID NO: 8 - protein, 103 aa, synthetic construct
  REGION 1..103: note = fp-6
IAALCGIVKSIDSDDSDYDYKGRGYCTNKGCRSGYNYFGNKGYCKYGEKSYTYNCNSYAGCCLPRNPYGKLKYYCTNKYGCPNNYYFYNNKGYYYLEHHHHHH

SEQ ID NO: 9 - protein, 199 aa, synthetic construct
  REGION 1..199: note = fp-151
AKPSYPPTYKAKPSYPPTYKAKPSYPPTYKAKPSYPPTYKAKPSYPPTYKAKPSYPPTYKPWSSEEYKGGYYPGNTYHYHSGGSYHGSGYHGGYKGKYYGKAKKYYYKYKNSGKYKYLKKARKYHRKGYKKYYGGGSSAKPSYPPTYKAKPSYPPTYKAKPSYPPTYKAKPSYPPTYKAKPSYPPTYKAKPSYPPTYKL

SEQ ID NO: 10 - protein, 171 aa, synthetic construct
  REGION 1..171: note = fp-131
AKPSYPPTYKAKPSYPPTYKAKPSYPPTYKAKPSYPPTYKAKPSYPPTYKAKPSYPPTYKPWADYYGPKYGPPRRYGGGNYNRYGRRYGGYKGWNNGWKRGRWGRKYYGSAKPSYPPTYKAKPSYPPTYKAKPSYPPTYKAKPSYPPTYKAKPSYPPTYKAKPSYPPTYKL

SEQ ID NO: 11 - protein, 175 aa, synthetic construct
  REGION 1..175: note = fp-353
PWADYYGPKYGPPRRYGGGNYNRYGRRYGGYKGWNNGWKRGRWGRKYYPWSSEEYKGGYYPGNTYHYHSGGSYHGSGYHGGYKGKYYGKAKKYYYKYKNSGKYKYLKKARKYHRKGYKKYYGGSSGSADYYGPKYGPPRRYGGGNYNRYGRRYGGYKGWNNGWKRGRWGRKYYGS

SEQ ID NO: 12 - protein, 187 aa, synthetic construct
  REGION 1..187: note = fp-153
AKPSYPPTYKAKPSYPPTYKAKPSYPPTYKAKPSYPPTYKAKPSYPPTYKAKPSYPPTYKPWSSEEYKGGYYPGNTYHYHSGGSYHGSGYHGGYKGKYYGKAKKYYYKYKNSGKYKYLKKARKYHRKGYKKYYGGSSGSADYYGPKYGPPRRYGGGNYNRYGRRYGGYKGWNNGWKRGRWGRKYYGS

SEQ ID NO: 13 - protein, 187 aa, synthetic construct
  REGION 1..187: note = fp-351
PWADYYGPKYGPPRRYGGGNYNRYGRRYGGYKGWNNGWKRGRWGRKYYPWSSEEYKGGYYPGNTYHYHSGGSYHGSGYHGGYKGKYYGKAKKYYYKYKNSGKYKYLKKARKYHRKGYKKYYGGSSGSAKPSYPPTYKAKPSYPPTYKAKPSYPPTYKAKPSYPPTYKAKPSYPPTYKAKPSYPPTYK

SEQ ID NO: 14 - protein, 60 aa, synthetic construct
  REGION 1..60: note = fp-1 variant
AKPSYPPTYKAKPSYPPTYKAKPSYPPTYKAKPSYPPTYKAKPSYPPTYKAKPSYPPTYK

SEQ ID NO: 15 - protein, 196 aa, synthetic construct
  REGION 1..196: note = fp-151 variant
AKPSYPPTYKAKPSYPPTYKAKPSYPPTYKAKPSYPPTYKAKPSYPPTYKAKPSYPPTYKSSEEYKGGYYPGNTYHYHSGGSYHGSGYHGGYKGKYYGKAKKYYYKYKNSGKYKYLKKARKYHRKGYKKYYGGGSSAKPSYPPTYKAKPSYPPTYKAKPSYPPTYKAKPSYPPTYKAKPSYPPTYKAKPSYPPTYK

SEQ ID NO: 16 - protein, 76 aa, synthetic construct
  REGION 1..76: note = mgfp-5
SSEEYKGGYYPGNTYHYHSGGSYHGSGYHGGYKGKYYGKAKKYYYKYKNSGKYKYLKKARKYHRKGYKKYYGGGSS